(19) United States
(12) Patent Application Publication
(10) Pub. No.: US 2020/0390503 A1
(43) Pub. Date: Dec. 17, 2020

(54) SYSTEMS AND METHODS FOR SURGICAL NAVIGATION AND ORTHOPAEDIC FIXATION

(71) Applicants: Carlos Quiles Casas, Badajoz (ES); Juan Antonio Constantino Cabrera, Badajoz (ES); Miguel Dominguez Melendez, Badajoz (ES)

(72) Inventors: Carlos Quiles Casas, Badajoz (ES); Juan Antonio Constantino Cabrera, Badajoz (ES); Miguel Dominguez Melendez, Badajoz (ES)

(21) Appl. No.: 16/899,075

(22) Filed: Jun. 11, 2020

Related U.S. Application Data
(60) Provisional application No. 62/860,905, filed on Jun. 13, 2019.

(51) Int. Cl. A61B 34/20; A61B 90/00; A61B 17/62; A61B 17/72

(52) U.S. Cl. CPC A61B 34/20; A61B 2034/105; A61B 17/62; A61B 17/72; A61B 2090/3983; A61B 2090/365; A61B 2090/3916; A61B 2090/367; A61B 2090/3945; A61B 2034/2051; A61B 2034/2055; A61B 2034/2063; A61B 2034/2048; A61B 2034/2059; A61B 2090/3762; A61B 2090/374; A61B 2090/378; A61B 90/37

(57) ABSTRACT

Systems and methods are described for the use of external fixators with intraoperative tracking of bone and/or attached devices and display of real-time information to the surgeon, for example for the treatment of bone deformities. Such systems and/or methods may include determining the deformity to be corrected; estimating the appropriate number, shape, and size of base members, transosseous fixations, and connecting elements to be used; suggesting optimal positions of said items relative to the bone segments; and calculating the necessary correction based on the final position of the attached external fixator components relative to the bone segments.

### RELATED APPLICATIONS

This application claims priority to U.S. Provisional Patent Application No. 62/860,905 by Carlos Quiles Casas, et al. on Jun. 13, 2019 and entitled, “Surgical navigation systems and methods for an orthopaedic fixation device,” the entirety of which is incorporated herein by reference.

### BACKGROUND

Patients with bone deformities suffer from a reduced quality of life. They may suffer from difficulties in standing, walking, or using limbs. Bone deformities can be congenital, or the result of a fracture that did not heal properly. Bone deformities may also occur at joints between two or more bones, which may cause the bones to be joined improperly, to be joined at an improper angle, to rub together and/or wear against each other, and so forth. These deformities can include axial, sagittal, or coronal plane deformities, translational or rotational deformities, malunion or nonunion deformities, or, in complex cases, more than one type of deformity. In at least some cases, therapeutic and/or surgical intervention may remedy such bone deformities. Intervention may include breaking, cutting, or re-shaping the deformed bone, setting the bone, and allowing the bone to regrow properly.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 illustrates a perspective view of a surgery navigation system, according to an embodiment.

FIG. 2 illustrates a method of surgery navigation, according to an embodiment.

FIG. 3 illustrates a virtual model including deformity parameters, according to an embodiment.

FIG. 4 illustrates another virtual model including deformity parameters, according to an embodiment.

FIG. 5 illustrates a virtual model including a base member position, according to an embodiment.

FIG. 6 illustrates a virtual model including selection of an origin of an osteotomy, according to an embodiment.

FIG. 7 illustrates a virtual model including mounting parameters, according to an embodiment.

FIG. 8 illustrates a method of adding a new fiducial, according to an embodiment.

FIG. 9 illustrates a perspective view of a target bone, fiducial, and virtual base member, according to an embodiment.

FIG. 10 illustrates a virtual trajectory of a transosseous fixator, according to an embodiment.

FIG. 11 illustrates a perspective view of the target bone with a real base member, according to an embodiment.

FIG. 12 illustrates insertion of a pin along the virtual trajectory, according to an embodiment.

FIG. 13 illustrates another perspective view of the target bone with the real base member, according to an embodiment.

FIG. 14 illustrates a perspective view of the target bone and the real base member without the fiducial attached to the target bone, according to an embodiment.

FIG. 15 illustrates the target bone with two attached base members, according to an embodiment.

FIG. 16 illustrates the target bone and two attached base members with struts extending between and connected to the two attached based members, according to an embodiment.

FIG. 17 illustrates a virtual model of the target bone with the two attached base members, where the virtual model shows progression of correction of the target bone deformity, according to an embodiment.

FIG. 18 illustrates a perspective view of a target bone fracture treated with a long plate, according to an embodiment.

FIG. 19 illustrates correction of the target bone using an intramedullary nail, according to an embodiment.

Corresponding reference characters indicate corresponding components throughout the several views of the drawings. Elements in the figures are illustrated for simplicity and clarity and have not necessarily been drawn to scale. For example, the dimensions of some of the elements in the figures may be exaggerated relative to other elements to help improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION OF THE INVENTION

Systems and methods for surgical navigation and orthopedic fixation as disclosed herein will become better understood through a review of the following detailed description in conjunction with the figures. The detailed description and figures provide merely examples of the various embodiments of systems and methods for surgical navigation and orthopedic fixation. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

Throughout the following detailed description, examples of various systems and methods for surgical navigation and orthopedic fixation are provided. Related elements in the examples may be identical, similar, or dissimilar in different examples. For the sake of brevity and clarity, related elements may not be redundantly explained in multiple examples. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an embodiment described as including A, B, C, and D is an embodiment that includes A, includes B, includes C, and also includes D. As used herein regarding a list, “or” forms a list of elements, any of which may be included. For example, an embodiment described as including A, B, C, or D is an embodiment that includes any of the elements A, B, C, and D. Unless otherwise stated, an embodiment including a list of alternatively-inclusive elements does not preclude other embodiments that include various combinations of some or all of the alternatively-inclusive elements. An embodiment described using a list of alternatively-inclusive elements includes at least one element of the listed elements. However, an embodiment described using a list of alternatively-inclusive elements does not preclude another embodiment that includes all of the listed elements. And, an embodiment described using a list of alternatively-inclusive elements does not preclude another embodiment that includes a combination of some of the listed elements. As used herein regarding a list, “and/or” forms a list of elements inclusive alone or in any combination. For example, an embodiment described as including A, B, C, and/or D is an embodiment that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted example embodiments.

Bone deformities are often treated with surgery. For example, surgeons may use metal implants to improve the geometry of a deformed bone. Inert metal implants may not be flexible in their ability to reform natural bone in something close to normal anatomical geometry. Surgeons may perform an osteotomy and attach an external fixator to support bone growth to correct the bone deformity. The Taylor Spatial Frame (TSF) is a commonly-used external fixator comprising rings interconnected by struts. After the osteotomy, the surgeon may insert pins through the superior and inferior sections of the bone. These pins are attached to external rings so that one ring is roughly perpendicular to the superior section of the bone, and the other ring is roughly perpendicular to the inferior section of the bone. The surgeon may attach adjustable struts to these rings so that the rings are held together by the struts. Each strut has a predetermined attachment point to each ring. Because the rings are each fixed to a section of bone, and because the rings are now joined by flexible struts, the bones can be moved with six degrees of freedom relative to each other.

After surgery to attach these rings and struts, a surgeon may take orthogonal X-rays of the apparatus on the patient's leg. The surgeon may make a number of measurements from the X-ray images, including distances and angles of both the bone and the rings and struts. The surgeon may then use the numerical measurements to calculate the bone correction needed and prescribe for the patient the length of each strut to be adjusted each day. Typically, daily adjustments will be made, realigning the sections of the bone at a rate that allows new bone to form, ultimately yielding natural bone in a geometry that comes close to normal anatomy and function. This system of two rings and six struts may be chosen for several reasons. First, the system allows a surgeon to move the two bone segments with six degrees of freedom relative to each other, thereby giving the surgeon the freedom to treat many types of deformities. The system may be strong enough to support body weight so that a patient can be ambulatory while healing occurs.

Such calculations are usually time-consuming and commonly rely on the assumptions that each ring is perfectly perpendicular to the bone segment to which it is attached. This may require a surgeon to spend extra time in the operating room to assure that each ring is perpendicular to each corresponding bone segment. If a ring is not perpendicular to its corresponding bone segment, error will be entered and the resulting prescription for strut adjustments will not be accurate. Other shortcomings of the procedure may include: the difficulty and lack of accuracy in using a ruler and protractor on an X-ray print-out, or a digital system not related to the prescription calculation program, to measure distances and angles; the amount of time involved in performing all the calculations needed to generate the patient prescription; and/or the surgical difficulty in positioning the external fixator exactly with respect to the patient's bone.

Various aspects and embodiments of the systems and methods for surgical navigation and orthopedic fixation described herein provide improved systems, methods and processes for the use of external fixators, for example to correct deformities of upper or lower limbs, for treatment of fractures, infections, bone defects, osteoarthritis, or any other pathology treatable with external fixators. The systems and methods may include navigation systems to plan and optimize treatment with external fixators. Treatment with external fixators may be associated with bone deformity corrections but may be used for many different chronic or acute pathologies. Implementations of the systems and methods described may allow easy and accurate planning, attachment, and measurements of bones and bone deformities, and may easily generate accurate prescriptions, such as for strut lengths for the bone correction treatment.

Systems and/or methods described herein may include determining a deformity to be corrected, the appropriate number, shape, and size of base members, transosseous fixations, as well as struts, rods or connecting elements to be used, as well as positioning of the external fixator components during surgery. During the application of an external fixator, the state of the external fixator components with respect to the bone may be assessed. Feedback may be provided on the state of the external fixator components with respect to the bone. The systems and methods described herein may provide more accurate real-time information than that obtainable by the conventional methods about deformities and their potential correction, and about positioning of devices and of transosseous fixations. The systems and/or methods described herein may also provide recommendations on device size, positioning and angulation, and other parameters relevant to achieving optimal results. The systems, methods, and/or processes may include databases of information or logic matrixes regarding tasks such as bone deformity estimation and correction estimation, in order to provide suggestions to the surgeon based on the actual deformity as defined from preoperative or intraoperative images.

Various systems and/or methods described herein may use computer capacity, including standalone and/or networked, to store data regarding spatial aspects of surgically related items and virtual constructs or references including body parts, implements, instrumentation, devices trial components, and/or rotational axes of body parts. Any or all of these may be physically or virtually connected to or incorporate any desired form of mark, structure, component, or other fiducial or reference device or technique which allows position and/or orientation of the item to which it is attached to be sensed and tracked, such as in three dimensions of translation, three degrees of rotation, in time, and so forth. Orientation of the elements on a particular fiducial may vary from one fiducial to the next so that sensors may distinguish between various components to which the fiducials are attached in order to correlate for display and other purposes data files or images of the components. Some fiducials may use reflective elements, and some may use active elements, either of which may be tracked by a sensor. Tracking of fiducials may be accomplished by an infrared sensor, emitter/detector or reflector systems including optic, acoustic or other wave forms (e.g. ultrasonic), shape based recognition tracking algorithms, video-based, mechanical, electromagnetic and radio frequency systems, inertial measurement, and so forth. An output of at least two sensors may be processed in concert to geometrically calculate position and orientation of the item to which the fiducial is attached.

Various items present during surgery, such as a surgical implement, instrumentation component, trial component, implant component or other device, may contain its own “active” fiducial such as a microchip with appropriate field sensing or position/orientation sensing functionality and communications link such as spread spectrum RF link, in order to report position and orientation of the item. Such active fiducials, or hybrid active/passive fiducials such as transponders may be implanted internally to the item and/or on an external surface of the item. Fiducials may also take the form of conventional structures such as a screw driven into a bone, or any other three-dimensional item attached to another item, position and orientation of such three-dimensional item able to be tracked in order to track position and orientation of body parts and surgically related items. Hybrid fiducials may be partly passive, partly active such as inductive components or transponders which respond with a certain signal or data set when queried by sensors according to the present invention.

A display may render virtual and/or real images to the surgeon. The display may be a video display. The video display may be mounted on a fixed or mobile support. The display may include an interactive tactile interface system. The display may be a stereoscopic display, offering either active or passive stereo vision, which may allow for a more accurate perception of depth. The display may be a head-mounted device. The head-mounted device may include headphones and a microphone for input and output of information in audio format. The head-mounted display may include one or more cameras to record and process real world images and combine them with virtual images to render both the real world images and the virtual images in real time. The head-mounted display may be an optical see-through or video see-through augmented reality device, which may include a stereo display, as well as its own positional tracking means and/or external tracking means (e.g. fiducials) to track the position of the head and/or eyes of the user, and/or to adapt the perspective of the rendered (virtual and/or real) images to the actual vision of the user. The augmented reality device may be enabled with gesture recognition, for example through motion tracking of the hands of the user. For example, the user may adjustment parameters of various virtual images displayed by the augmented reality device by hand gestures, and so forth. More than one type of display may be used by the same or different users in an operative (e.g. surgical) setting.

A computer may calculate and store reference axes of body components such as in deformity correction, for example, the mechanical axis of the tibia and/or femur. From these axes, the position of bone segments and devices may be tracked so that the surgeon may locate base members optimally. During virtual trials of components, for example for deformity correction, feedback may be provided on the size and position for the specific anatomical area and in a range of motion. Accurate information may be provided about which shape, size, and precise location and angulation to select for base members and transosseous fixations. Modifications to devices, fixations, their positioning, and other techniques to achieve optimal treatment may also be provided based on the information determined by tracking various elements during surgery. Databases of information may be provided regarding tasks such as estimation of deformity parameters, device stability, or clinical cues, in order to provide automatic (e.g. on-demand) suggestions to the surgeon, such as during surgery.

Connections between structures described herein may be direct operative connections or indirect operative connections through intermediary structures. A set of elements may include one or more elements. A recitation of an element may be understood to refer to at least one element and perhaps more. A plurality of elements may include at least two elements. Unless otherwise required, any described method steps need not be necessarily performed in a particular illustrated order. A first element (e.g. data) derived from a second element may encompass a first element equal to the second element, as well as a first element generated by processing the second element or other data. Azimuthal rotation of a ring may refer to rotation of the ring about an axis passing through a center of the ring and normal to a major plane of the ring. Axial rotation of a ring may refer to a non-azimuthal rotation of the ring, i.e. to a rotation about an axis different from the axis passing through the center of the ring and normal to the major plane of the ring. An axial rotation may be performed about an axis lying in the major plane of the ring, about a point along the ring or tangent to the ring, or about another axis. Making a determination or decision according to a parameter may encompass making the determination or decision according to the parameter or according to other data. Unless otherwise specified, an indicator of some quantity/data may be the quantity/data itself, or an indicator different from the quantity/data itself.

Computer programs described in some embodiments of the present invention may be stand-alone software entities or sub-entities (e.g., subroutines, code objects) of other computer programs. Computer readable media encompass non-transitory media such as magnetic, optic, and semiconductor storage media (e.g. hard drives, optical disks, flash memory, DRAM), as well as communications links such as conductive cables and fiber optic links. According to some embodiments, the present invention provides, inter alia, computer systems comprising hardware (e.g. one or more processors and associated memory) programmed to perform the methods described herein, as well as computer-readable media encoding instructions to perform the methods described herein.

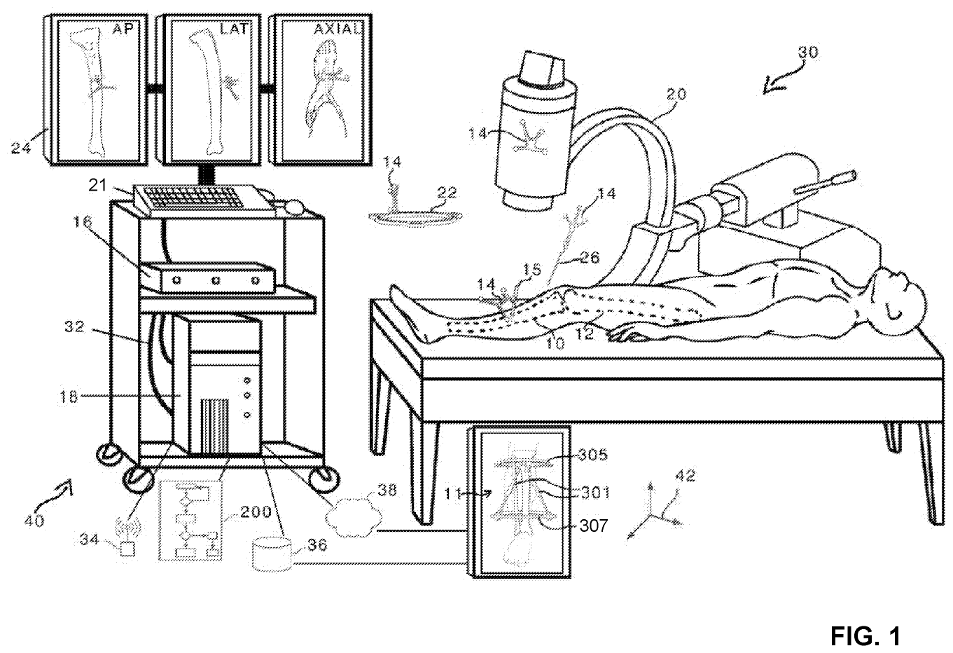

In reference to FIGS. 1, 3-7, 9-17, the external fixator device (e.g. the corrective device 11) may include two circular base members 305, 309, and/or adjustable length struts 301 interconnecting the two base members 305, 309. The base members 305, 309 may be interconnected by six struts 301. The struts 301 may be attached to the base members 305, 309 by split-ball connectors 303. The detailed description of the systems and methods provided herein may relate to hard tissue deformities, such as bone deformities, and may be described more specifically in relation to tibial deformities. Although the following systems and/or methods may be described in such terms, it may be understood how to apply these systems and/or methods in other ways, including using devices such as Taylor Spatial Frame or another hybrid external fixator, an Ilizarov or a similar circular fixator, or any other type of external fixator or combination thereof, which can be used for treatment of any kind of chronic or acute pathology of bone or soft tissues.

A single bone technique may be used to correct congenital deformities and acquired or post-traumatic malunion or nonunion deformities, which may affect one long bone or two or more bones fused into one. The single bone technique may be applied as a part of a technique involving multiple bones, for example a simultaneous correction of tibial and femoral deformities, wherein each bone may be treated independently and simultaneously.

FIG. 1 is a perspective view showing one version of a setting in accordance with the single bone technique in which surgery on a single bone, in this case a tibial deformity correction, may be performed. Some of the features in FIG. 1 may be the same as or similar to some of the features in the other FIGs. described herein as noted by same and/or similar reference characters, unless expressly described otherwise. Additionally, reference may be made to features shown in any of the other FIGs. described herein and not shown in FIG. 1. Systems and processes described herein may enable tracking of various bone segments such as the target bone 10 with fiducials of the sort described above or any other sort that may be implanted, attached, or otherwise associated physically, virtually, or otherwise. Fiducials 14 may be structural frames. The fiducials 14 may include reflective elements and/or LED active elements for tracking using one or more sensors. For example, the elements may be tracked using stereoscopic infrared sensors, operating in concert, for sensing, storing, processing and/or outputting data relating to (“tracking”) position and orientation of the fiducials 14 and/or components or body parts such as the target bone 10 to which they are attached or otherwise associated. A pin 15, such as a Steinmann pin, a half-pin, or any other kind of metal rod with or without a screw at its end, or any other device may hold the fiducial 14 firmly to the target bone 10 and/or soft tissues around the target bone 10.

The tracking system 40 may include the computer 18 (e.g. a computer with a processing, memory, communication, and/or display functionality) and one or more position sensors 16 adapted to sense the position of the fiducials 14 remotely. The position sensors 16 may include cameras, such as CCD cameras, CMOS cameras, and/or optical image cameras, magnetic sensors, radio frequency sensors, or any other sensors adapted to detect and/or track the position of the fiducials 14. The position sensors 16 may be mounted on a stand, on a mobile cart, and/or by other means whereby the fiducials 14 are detectable and/or trackable by the position sensors 16. The tracking system 40 may include one position sensor 16, two position sensors 16, three position sensors 16, and so forth. The cameras may be adapted to capture a view of the fiducials 14 from a different position. The computer 18 may include software and/or hardware having computing instructions adapted to calculate the location of trackable devices relative to the coordinate system 42 (e.g. a common coordinate system for tracked elements) by triangulation of different views of the trackable devices (e.g. the fiducials 14, operative instruments using during surgery, base members 305, 309, and so forth). Items 22 such as trial base members or instrumentation components may be tracked in position and orientation relative to tracked body parts 10 using fiducials 14.

The computer 18 may include processing functionality, memory functionality and/or input/output functionality on a standalone or distributed basis, via any of a variety of standards, architectures, interfaces and/or network topologies. The computer 18 may include input/output devices, such as a keyboard and mouse 21 and one or more display monitors 24. The display monitors 24 may include one or more data communication devices, such as communication cables 32 and/or wireless communication devices 34. Foot pedals or another convenient interface may be coupled to the computer 18 as may any other wireless or wireline interface to allow the surgeon, nurse, and/or other user to control and/or direct the computer 18. Controlling and/or directing the computer 18 may, for example, include capturing position/orientation information when a component is oriented or aligned properly. The computer 18 further may have access to one or more databases 36 for storing various data. The computer 18 may include a data connection to a larger computer network 38, such as via the internet, wide area network, local area network, and/or other computer networking means. Additional and/or alternative and/or fewer hardware and programming components may also be implemented as part of the computer 18. Databases 36 and/or network 38 may be used to select the appropriate anatomic location such as target bone 10, specific corrective device 11, and/or pathology (for example, tibia bone deformity) among stored pre-set configurations, for example by the surgeon, nurse, or other user in a touch screen monitor.

The computer 18 may process, store and output on a display 24 various forms of data which correspond in whole or part to the target bone 10 and/or items 22. For example, the surface structure of the target bone 10 may be shown at various perspectives, based on intraoperative images, for example anteroposterior (AP) and lateral-medial (LAT) fluoroscopic images of the tibia. These images may be obtained using an imaging device 20, such as a C-arm, which in some embodiments may be attached to a fiducial 14. The bone segments, such as a single tibial segment 10, may have a fiducial 14 attached.

When fluoroscopy images are obtained using the imaging device 20 with the attached fiducial 14, a position/orientation sensor 16 may “see” and/or track the position of the fluoroscopy head as well as the positions and orientations of the target bone 10. The computer 18 may store the fluoroscopic images with this position/orientation information, thus correlating position and orientation of the fluoroscopic image relative to the relevant body part or parts. When fluoroscopy images are obtained using the imaging device 20 without fiducials 14, pre-defined anatomical landmarks represented by points on images of the target bone 10 and markers forming part of the fiducial 14 may be designated, selected, registered, and/or otherwise made known to the tracking system 40, such as via intraoperative X-rays. The tracking system 40 may include an image calibration system and/or a ground truth reference device attached to the imaging device 20 and/or the target bone 10, or interposed between them, such as a C-arm image calibrator and/or radio-opaque phantoms. Anatomic or mechanical axes may be assessed preoperatively and/or intraoperatively using long films including hip, knee, and ankle. This selection may be input manually by the user, or automatically by the computer 18 with or without further adjustments by the user.

The computer 18 may register preoperative images, which may include selected landmarks and/or surgical planning, with intraoperative images. The computer 18 may register the obtained intraoperative images to a three-dimensional model, for example a 3D volume reconstruction of a preoperative or an intraoperative computed tomography (CT) scan, or a shape model of the target bone 10 constructed based on 2D or 3D images, or on the preselected anatomical data that the user inputs to the system 40. A 3D volume may be obtained from an intraoperative image obtained with a CT scan, cone-beam CT, or similar intraoperative imaging device 20 attached to a fiducial 14. The 3D volume may be automatically registered to the current position of the target bone 10. In embodiments, a combination of preoperative or intraoperative images may be registered using an algorithm for image registration. The computer 18 may register preoperative and/or intraoperative images of soft tissues surrounding the target bone 10. The preoperative and/or intraoperative images may include volumetric reconstruction of a CT or magnetic resonance imaging (MRI) scan, surface reconstruction with a three-dimensional scanner device, photogrammetric reconstruction through a video camera (which may be part of the position/orientation sensor 16), and so forth. The computer 18 may store the estimated soft tissue volume, for example by automatically estimating areas based on image classification algorithms applied to intraoperative fluoroscopic images, based on input of the surgeon of the estimated diameter of the leg, based on the use of a probe 26 or other tracked item 22 to fit the size of soft tissues (e.g. fitting one or more tracked base members to a certain area, and indicating to the computer 18 to store the diameter of the leg for each segment), and so forth.

The stored image data set of the target bone 10 rendered on the display 24 may be formed by preoperative and/or intraoperative images, such as fluoroscopic images of bones, and/or by computer generated images of the corresponding three-dimensional (3D) models together with virtual constructs (e.g. 2D and/or 3D virtual representations) and references of items 22 such as implements, devices, instrumentation, components, and any other object used in connection with surgery for navigation, assessment and other purposes. When the target bone 10 and/or corresponding fiducial 14 move, the computer 18 may automatically and correspondingly sense the new position of target bone 10 in space and may correspondingly move tracked items 22 such as devices, instruments (like drill and/or saw motors), components, and references on the display 24 relative to the image of target bone 10. Similarly, the image of the body part may be moved, or both the body part and such items may be moved, or the on-screen image may otherwise be presented to suit the preferences of the surgeon or other users and carry out the perspective rendering of real and/or virtual images that is desired by the surgeon or other users. Similarly, when an item 22 such as a ring, half-ring, or any other type of base member that is being tracked moves, its image may move on display 24 so that the monitor shows the item 22 in proper position and orientation on the display 24 relative to the target bone 10. For example, a ring may appear on the display 24 in proper or improper alignment with respect to the mechanical axis and other features of the target bone 10, as if the surgeon were able to see into the body in order to navigate and position said ring properly.

The computer 18 may store data relating to configuration, size, and other properties of items 22 such as devices, instruments, references, and other objects used for surgery or during preoperative and postoperative treatment. When those are introduced into the field of position/orientation sensor 16, the computer 18 can generate and display said items 22 overlaid or otherwise merged with the fluoroscopic images of the body part 10, and with computer generated images of other items 22 such as devices, instruments, references, and other objects used for navigation, positioning, assessment and other uses.

The computer 18 may render in the display 24 a virtual realistic representation of the relationship between the body part and items 22 whose position is tracked, and/or a schematic and/or mathematical representation of such relationship, for example the angles and distances between bone segments, between items 22, or between bone segments and items 22.

The computer 18 may track any point in a field of sensing of the position sensor 16 such as by using a designator or a probe 26. The probe may contain or be attached to a fiducial 14. The surgeon, nurse, or other user may touch with the tip of probe 26 a point such as a landmark on a bone structure and instructs the computer 18 to note the landmark position. The position/orientation sensor 16 may “see” the position and/or orientation of fiducial 14, and/or may “know” where the tip of probe 26 is relative to that fiducial 14. The computer 18 may calculate and/or store, and may display on monitor 24, the point or other position designated by probe 26 when a command is given. The point or other position may be displayed automatically or when prompted and/or may be displayed in a selected color or in an automatically-assigned color. The probe 26 may be used to designate landmarks on bone structure in order to allow the computer 18 to store and/or track, relative to movement of the bone fiducial 14, virtual or logical information such as mechanical axis 28, medial lateral axis 30, anterior/posterior axis 32, and axial rotational axis of the target bone 10 and other body parts in addition to any other virtual or actual construct or reference. The probe 26 may be used to select, designate, register, or otherwise make known a point or points on the anatomy or other locations by placing probe 26 as appropriate and signaling or commanding the computer 18 to note the location of, for instance, the tip of probe 26, for example by “painting” the outer surface of the leg and storing the registered outer soft tissue surface relative to the tracked target bone 10. The probe 26 may be used to test the registration accuracy by selecting the position of these anatomical point or points or other locations and comparing them with the corresponding points previously selected by the user or automatically identified by the tracking system 40 in the image data set.

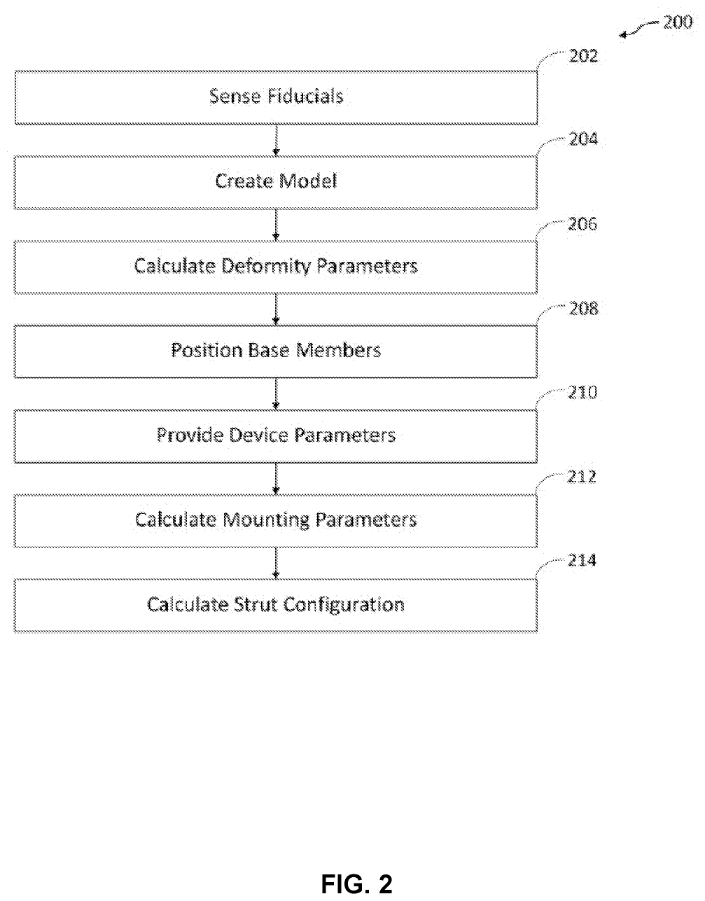

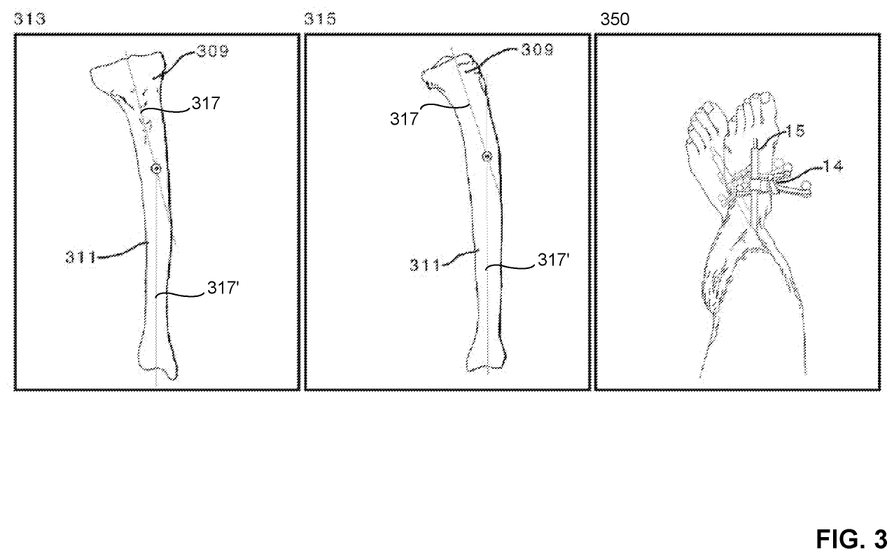

As illustrated in FIG. 2, the method 200 (i.e. the navigation procedure), when implemented together with other components of the navigation system 30, may enable the navigation system to track the position of the tracked bone or bones 10 and additional tracked items 22, relative to the coordinate system 42. The navigation procedure may be implemented in software programming code accessed and/or implemented by the computer 18, for example from the database 36 and/or from a remote source such as via the internet at 38 and may include one or more ASIC configurations. Some of the features in FIG. 2 may be the same as or similar to some of the features in the other FIGs. described herein as noted by same and/or similar reference characters, unless expressly described otherwise. Additionally, reference may be made to features shown in any of the other FIGs. described herein and not shown in FIG. 3

The method 200 may include obtains the initial locations of the fiducials 14, such as the fiducials attached to the target bone 10 and to the imaging device 20 which obtains the intraoperative images (e.g. fluoroscopic images, and so forth) (block 202). The initial locations may be in the form of coordinates relative to the coordinate system 42 or some other arbitrary coordinate system, for example, defined relative to the fiducials 14. The method 200 may include creating an initial model of the target bone 10 from the initial location of the fiducials 14 and selected and registered anatomic landmarks (block 204).

As further illustrated in FIG. 3, the method 200 may include calculating a deformity parameter (block 206). Some of the features in FIG. 3 may be the same as or similar to some of the features in the other FIGs. described herein as noted by same and/or similar reference characters, unless expressly described otherwise. Additionally, reference may be made to features shown in any of the other FIGs. described herein and not shown in FIG. 3. The surgeon may measure, using the computer 18, the anteroposterior view 313 and lateral-medial view 315 of the target bone 10, based on pre-defined anatomical landmarks. A common axis (anatomic and/or mechanical) may be identified for the bone segments 309, 311 for use in making the measurements, which may be fully automated by the computer 18 or adjusted by the surgeon. The surgeon may perform a clinical exam of the target bone 10 with attached fiducial 14, for example to estimate internal-external rotation range and precise axial rotation related to each position of the fiducial 14. Data and/or information collected by the surgeon during the clinical exam may be stored by the computer 18. These measures may yield deformity parameters, which may be determined with following measurements of the position of the first bone segment 309 relative to the second bone segment 311 as determined from radiographs and a clinical examination. The measures may yield, for example, six deformity parameters, including: 1) anterior-posterior (AP) displacement as seen on the lateral (LAT) view 313; 2) LAT displacement, as seen on the AP view 315; 3) axial displacement, as seen on either the LAT or AP view 313, 315; 4) AP angulation, as seen on the LAT view 313; 5) LAT angulation, as seen on the AP view 315; and/or 6) axial rotation, as may be determined through clinical examination. Calculating the deformity parameter may include determining an origin at the deformity site that will act as a convenient reference point, preferably at the same level that the AP and LAT displacements are measured. The computer 18 may offer an automated process, for example fully automated registration of intraoperative with preoperative images, which may include pre-planned deformity parameters and correction planning, or registration of specific anatomical point or points, or of the obtained mechanical and/or anatomical axes, or any other registration method applied to preoperative plans and/or images, in all cases with or without manual correction by the user of the final registration obtained. An algorithm may be used to obtain an automated intraoperative 2D to preoperative 3D image registration, intraoperative 3D to preoperative 3D registration, and/or another combination of preoperative and/or intraoperative imaging.

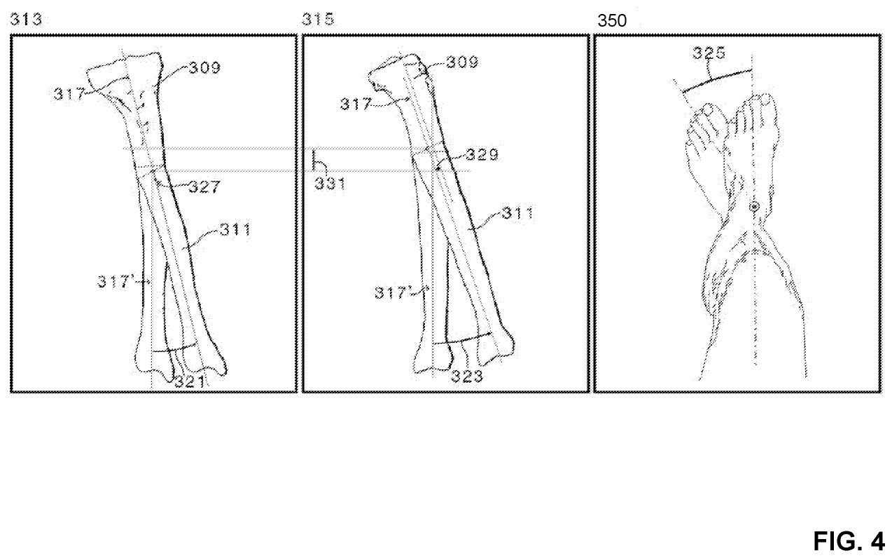

One fragment of the target bone 10 may be treated as a stationary reference to establish a frame of reference for the target bone 10, such as in the coordinate system 42. The other fragment may be treated as moving or deformed. A deformity of the distal fragment may be characterized with respect to a proximal fragment, i.e., the proximal fragment may be the reference fragment, and the distal fragment may be the moving fragment. A method of using the fixator 14 may be used when the distal fragment is considered the reference fragment and the proximal fragment is considered the moving or deformed fragment. This may be useful in proximal tibial nonunions or malunions with a short proximal fragment (e.g. as illustrated in FIGS. 1, 3-7, 9-16). The location of the attachment of the proximal base member (using the joint surface and fibular head as landmarks) may be more precisely determined in preoperative planning and in surgery than the level of attachment of the distal base member on the longer distal fragment. This may enable the navigation system and the surgeon to fully characterize the deformity even though the radiographs may be too short to include the level of attachment of the distal base member. Limitation of intraoperative imaging for bone segment selection as reference may be eliminated by linking, in the preoperative and/or intraoperative setting, the fiducial 14 with the target bone 10 into a stable fiducial-bone relationship. Eliminating the limitation eliminates a need for repetitive intraoperative fluoroscopic imaging of parts of the target bone 10. Instead, linking the fiducial 14 with the target bone 10 may provide the surgeon with accurately registered and readily available image data of the target bone 10, such as intraoperative fluoroscopic images and/or a full 3D model of the target bone 10. In FIGS. 3-4, a selection of the moving bone segment 311 as the distal fragment is nevertheless made, for simplicity purposes and purposes of illustration.

FIGS. 3 and 4 illustrate how the deformity parameters are determined for a deformed tibia. The description of deformity parameters may be made in accordance with the common orthopaedic convention regarding anatomical planes. Another convention may be used that departs from common orthopedic convention. Once a standard is selected, consistent application of the convention may be employed in the navigation system. As an example, the proximal fragment 309 may be the reference fragment, and the distal fragment 311 may be the moving fragment. AP 313, LAT 315, and axial 350 views of the tibia are shown, which may be three-dimensional or two-dimensional (2D) views including virtual and/or real images. The centerline 317 of the reference fragment 309, and the centerline 317′ of the deformed moving fragment 311 are drawn. Both centerlines 317, 317′ may extend along the common axis of the corrected bone. The AP angulation is indicated by the arrow 321 and the LAT angulation is indicated by the arrow 323, and may be determined by using traditional methods to measure the divergence of the centerlines 317, 317′ drawn in each fragment 309, 311, with help from the computer 18, either in preoperative planning or intraoperatively, which may include further adjustments by the surgeon. Axial rotation (internal or external rotation) may be assessed clinically with the help of attached fiducials 14, with or without help from special films, preoperatively or intraoperatively. The axial rotation, as indicated by the arrow 325, may be the amount of rotation about the centerline of the bone from its normal position. Translation deformity parameters (i.e. displacement) may be determined as the perpendicular distance from the reference fragment's centerline 317 to the moving fragment's centerline 317′ at the level of the origin, which may be the interior end of the moving fragment 311. The AP translation may be indicated by the arrow 327, and may be almost zero, while the LAT translation is indicated by the arrow 329. The axial translation may be measured on either the AP or the LAT radiograph, and may be the distance between the interior ends of the fragments 309, 311 measured along the reference fragment centerline 317, indicated in FIG. 4 by the arrow 331. The signs for the deformity parameters may be based on the coordinate axes with the right-hand rule as selected conventions. Some of the features in FIG. 4 may be the same as or similar to some of the features in the other FIGs. described herein as noted by same and/or similar reference characters, unless expressly described otherwise. Additionally, reference may be made to features shown in any of the other FIGs. described herein and not shown in FIG. 4.

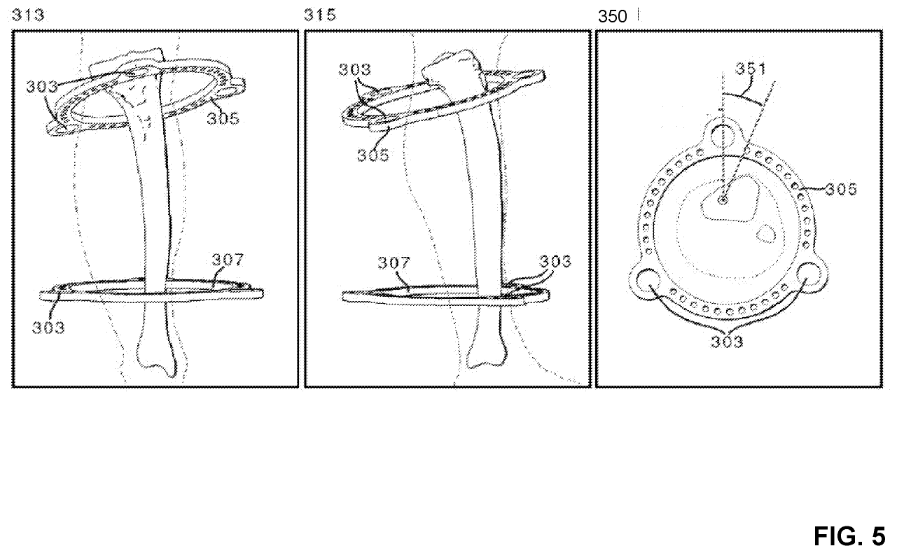

The method 200 (i.e. the navigation procedure) may include selecting (e.g. the surgeon may select, the computer 18 may select, and so forth) an appropriate size corrective device (e.g. external fixator, internal fixator, and so forth) and a position for the corrective device using real or virtual trials as illustrated in FIG. 5 (block 208). Some of the features in FIG. 5 may be the same as or similar to some of the features in the other FIGs. described herein as noted by same and/or similar reference characters, unless expressly described otherwise. Additionally, reference may be made to features shown in any of the other FIGs. described herein and not shown in FIG. 5. The real or virtual trials may be based image data of the target bone 10 (e.g. intraoperative fluoroscopic images or a 3D model/shape of the target bone) with surrounding soft tissues rendered on the display 24. Virtual trials may include further clinical examination and the use of a base member 22 with attached fiducial 14. Once a corrective device size has been selected for both the first base member 305 and the second base member 307, device parameters may be provided (block 210). The device parameters may include: 1) the effective diameter of the first base member 305, 2) the effective diameter of the second base member 307, and 3) the initial neutral length of the struts 301 for both base members 305, 307 in the preferred position. The effective diameter of a base member 22 may be the diameter of a circle that substantially intersects the connectors 303 associated with such base member. The base members 22 may be circular, and therefore their effective diameters may be the actual diameters of the base members 22. Other base member shapes may be used in the external fixator device. While this example shows the use of two base members of a circular shape and specific transosseous and connecting elements for simplicity purposes, alternative configurations with multiple basic or intermediate members, and many potential combinations of transosseous and connecting elements to fix these base and intermediate members between them and to bone may be used for different devices, pathologies, and anatomic locations.

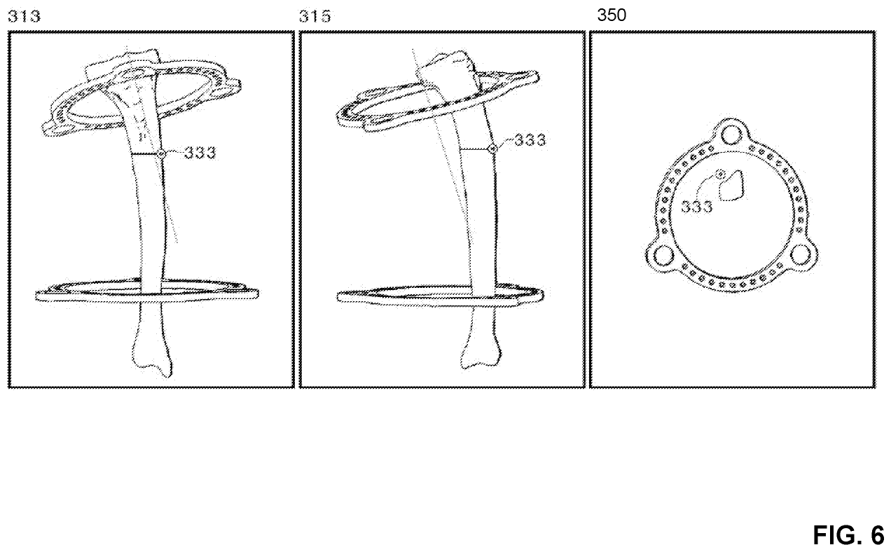

FIG. 6 illustrates the origin 333 for the example followed in previous illustrations. Some of the features in FIG. 6 may be the same as or similar to some of the features in the other FIGs. described herein as noted by same and/or similar reference characters, unless expressly described otherwise. Additionally, reference may be made to features shown in any of the other FIGs. described herein and not shown in FIG. 6. Since the deformity has significant angulation, a point on the convex cortex of the interior end of the moving fragment may be chosen as the origin 333 to prevent a compressive hinge and excessive preload on pins and wires. The origin 333 may be placed at the center of the moving fragment rather than the convexity of the deformity. The computer 18 may offer options for positioning the origin 333 in the preoperative setting or in real time during surgery. The options offered by the computer 18 and/or their order of preference may depend on previous selections and may be selected and further adjusted manually by the surgeon. Rotation at the convex cortex may be necessary for correction of congenital deformities, malunions, and stiff nonunions that may include minimal or no lengthening. Too much impaction and over constraint at the convex cortex may result in excessive preload on pins and wires and under-correction of the deformity.

The surgeon may adjust the anticipated location of the devices relative to the bone 10 and soft tissues, predetermining an appropriate location on the first bone segment 309 for attachment of the first base member 305 using input capabilities of the computer 18. The method 200 may include calculating (e.g. by the computer 18) the relative position of the deformity with respect to the corrective device (block 212). The calculation may provide mounting parameters or device eccentricities, such as 1) the vertical distance from the first base member 305 to the origin; 2) the horizontal displacement of the origin from the centerline of the corrective device 11, whereby the horizontal displacement consists of a) anterior-posterior displacement and b) lateral-medial displacement; and/or 3) a predetermined orientation of the corrective device 11 and the amount the second bone fragment 311 is rotated about its axis from its correct position. The first base member 305 may be considered to be the moving base member and the second base member 307 may be considered to be the stationary reference base member.

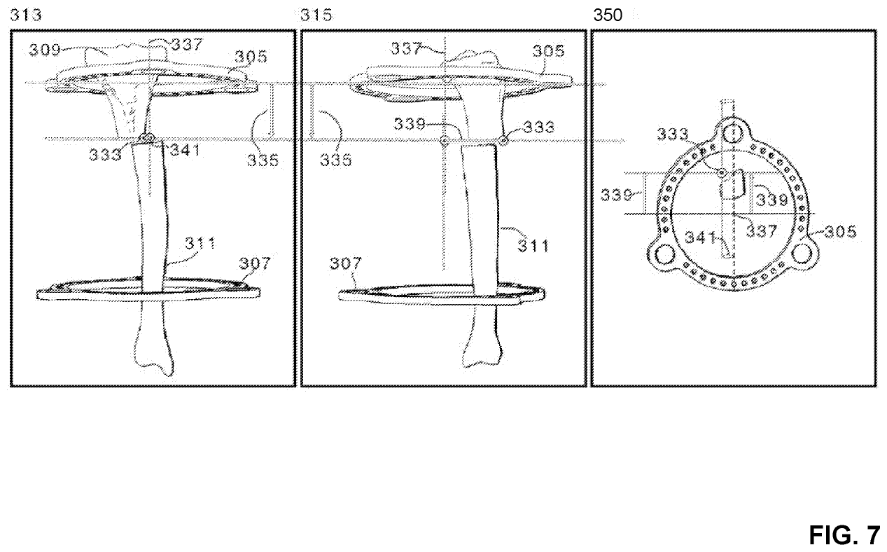

Turning now to FIG. 7, after the origin 333 is selected, the computer 18 may characterize the position of the corrective device 11 (including base members 305, 507) relative to the origin 333 and render it to the display 24. Some of the features in FIG. 7 may be the same as or similar to some of the features in the other FIGs. described herein as noted by same and/or similar reference characters, unless expressly described otherwise. Additionally, reference may be made to features shown in any of the other FIGs. described herein and not shown in FIG. 7. The user (e.g. the surgeon) may make further adjustments. The distal fragment and base member may be considered the reference fragment and base member, and the proximal fragment and base member may be considered moving. Specifically, the second bone fragment 311 and the second base member 307 may be considered the reference points, and the first base member 305 and the first bone fragment 309 may be considered the moving components. The computer 18 may define axial eccentricity as indicated by the arrow 335, which may be the measurement of length parallel to the device centerline 337 from the level of the moving base member 305 to the origin 333. The tibia may be located anterior to the geometric center of the base member, and the computer 18 may automatically measure and render in the display 24 in the LAT view 315 the distance from the centerline 337 of the corrective device 11 to the origin 333 within a plane parallel to the moving base member 305. This distance may be the lateral eccentricity as indicated by arrow 339. In the AP view 313, the computer 18 may measure the distance from the centerline 337 of the corrective device 11 to the origin 333 within a plane parallel to the moving base member 305. This distance may be the AP eccentricity as indicated by the arrow 341. Accordingly, the position of the bone with respect to the corrective device 11 may be anticipated.

In order to characterize the rotational position of the corrective device 11 relative to the skeleton, an orientation of the corrective device 11 relative to the skeleton may be adopted to provide a frame of reference. The connector 303 of the proximal base member 305 (i.e., the master connector) may be placed between struts 301 located anterior. The axial view 350 illustrated in FIG. 5 shows the rotary eccentricity for a tibia. The rotary eccentricity is indicated by line 351 and may be determined clinically as the amount of rotation of the bone relative to the corrective device 11 from the orientation adopted as the frame of reference.

The method 200 may include calculating a strut configuration (block 214). The strut configuration may include strut lengths. The strut lengths may configure the hexapod circular external fixator to mimic the deformity if the corrective device 11 is places on the bone segments 309, 311 at the predetermined appropriate location. Parameters for the configuration may be stored by the computer 18 and input to a calculator program based on a deformity equation. The computer 18 may calculate the strut lengths using the deformity equation. When mimicking or mirroring a deformity, one of the base members 305 may be rotated and/or translated. The corrective device 11 may initially be placed in any selected position, and a rotational motion may be first applied. Thereafter a translation motion may be applied. The deformity equation may include a rotation component R! and a translation component T!, for example as described for the Taylor Spatial Frame in U.S. Pat. No. 5,728,095 (which is hereby incorporated by reference in its entirety). The rotation and/or translation components may be dependent on the specific needs of the patient and the specific corrective device 11 used. The computer 18 or a calculator may be programmed to calculate the strut lengths based on the input of variables. The variables may include the device parameters, the deformity parameters, and the eccentricities. Because accurate parameters may be obtained by tracking the base members attached to each bone segment, various information may be obviated as unnecessary for correcting the bone deformity. The information may include a predetermined position of the corrective device 11 relative to the bone segments 309, 311 in the preoperative setting to facilitate deformity correction from or to a neutral position and/or postoperative manual or computer-based calculations determined by anatomic and device landmarks selected on X-rays.

Various blocks of the method 200 (i.e. the navigation procedure) may be executed before the surgeon commits to the definitive surgery. Blocks 204, 206, 208, 210, and/or 212 may be performed during setup of the navigation procedure. A virtual navigation procedure may be performed, and steps repeated, as the surgeon performs the actual surgery involving the attachment of base members 305, 307 to the pre-selected position in bone segments 309, 311. The virtual navigation procedure may be started automatically by the computer 18 as it detects changes through input from the navigation system 30, or manually through input from the user, that the initial conditions (such as deformity parameters or soft tissue considerations) have been modified, that the bone-fiducial relationship has been altered, or that pre-planned positions for devices have changed.

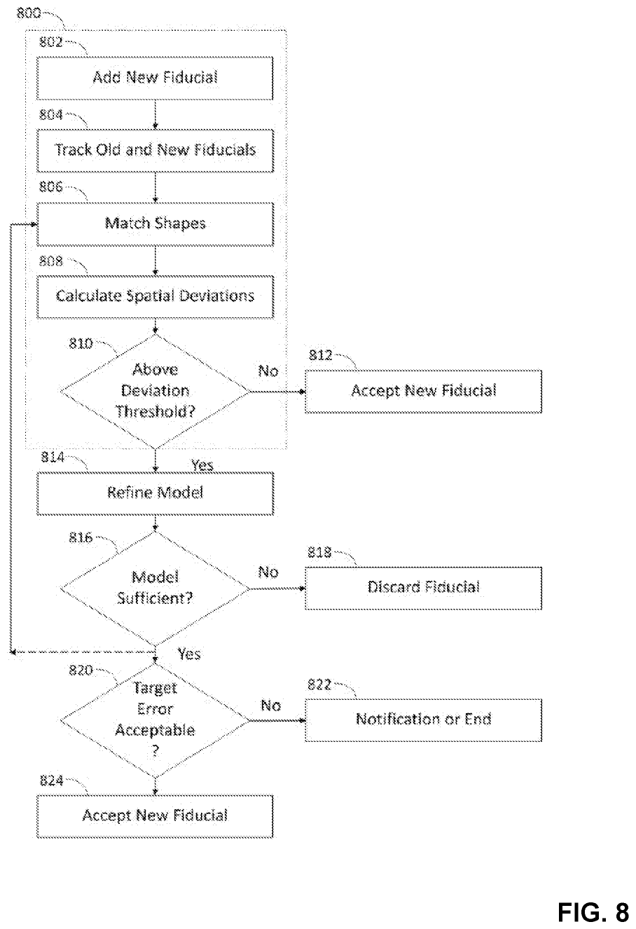

Block 202, where changes to the bone-fiducial relationship are sensed, may be done iteratively during the whole navigation procedure and, depending on calculated changes to that bone-fiducial relationship, blocks 204 and 206 may be repeated. In FIG. 8, these steps may be performed as part of the block 800 when a new fiducial attached to a bone segment is added to the tracking system 802. Some of the features in FIG. 8 may be the same as or similar to some of the features in the other FIGs. described herein as noted by same and/or similar reference characters, unless expressly described otherwise. Additionally, reference may be made to features shown in any of the other FIGs. described herein and not shown in FIG. 8. The new fiducial may be sensed by the position sensors 16 of the tracking system 40 together with the other fiducial or fiducials 14 attached to same bone segment, and their position relative to the selected coordinate system 42 may be obtained (block 804). A model of the bone segment may be created based on the position of the new fiducial relative to the older ones (block 806). Image data of the target bone 10 (e.g. the 3D model/shape) may be matched with older fiducials by an algorithm (such as a least squares analysis to find the best fit). Different positions of the target bone 10 according to movements performed by the surgeon, such as flexion-extension of the hip, knee, and ankle, knee rotation, and hip abduction-adduction, may be used to test different positions of the target bone 10 in the coordinate system subject to potential soft tissue deformations and collisions. As the movements are performed, the computer 18 may store the different shapes obtained at each position, for example in pairs of shapes if there is only one old and one new fiducial to compare. The computer 18 may calculate the spatial deviation of the shape pairs obtained for each particular position using a selected algorithm, which may include the distance and/or direction of each deviation at each particular position (block 808). The computer 18 may estimate whether this deviation is above a selected threshold, which may be defined as a static or dynamic value and may be adjusted by the surgeon (block 810). If the spatial deviations are not above the established threshold, the new fiducial may be accepted (block 812). If they are above the selected threshold, the model may be refined for the different positions automatically, with or without adjustments by the surgeon (block 814). A visual comparison may be displayed of differences between the old and new shapes rendered overlaid or side by side in the display 24. Numeric values for each fiducial and each position of the tibia, including potential causes (such as increase of variability during flexion-extension movements, potentially due to soft tissue pressure) may be displayed. The consequences of accepting the new shape with the estimated deviations, in terms of accuracy and precision of calculations of device parameters, mounting parameters, and strut configuration may be displayed. A refined model may be thus created by correcting the position of the new and/or old fiducials, for example by compensating for deformations of the fiducial attachments, such as pressure of the soft tissues on the pins holding the old and/or new fiducials. A refined model may be obtained by combining the models according to an estimated best working fiducial, for the new and/or old fiducial to work alone. For example, the new fiducial 14 on the first base member 305 may be slightly moved in flexion when the base member 305 touches the posterior aspect of the thigh, while the initial fiducial 14 directly attached the target bone 10 may be stretched during extension of knee and/or ankle, due to changes in soft tissues of the calf. The tracking system 40 may obtain the best combination of fiducials 14 for each lower limb position, and/or refine a single common model for each fiducial 14 to work independently from each other, based on the recorded data when both fiducials 14 are still attached.

After refining the model, the navigation procedure may include one or more sufficiency checks. For example, the navigation procedure may include determining whether the refined model is still sufficient for use in the navigation procedure (block 816). If the step of refining the model includes removing one or more fiducials from the refined model, for example due to a high variability of deviations (in direction and/or distances) indicative of a non-fixed position relative to the bone segment, the computer 18 may determine if the remaining set of fiducials in the refined model includes the new fiducial. If the new fiducial is not included, the new fiducial may be discarded (block 818). The steps of matching the old and new models, calculating spatial deviations, and refining the model may be iteratively repeated, including old fiducials, until either no further fiducials are removed from the set forming the refined model, or the refined model includes fewer than a predefined minimum number considered acceptable before re-setting or re-registering the navigation system 30. The navigation system 30 may be set to at least one fiducial per bone segment. The computer 18 may calculate an averaged spatial deviation of up to all of the fiducials defining the refined model from the spatial deviations. The navigation procedure may evaluate the new fiducial (e.g. block 816 where the refined model is evaluated). The navigation procedure may include discarding the new fiducial when the averaged spatial deviation exceeds a selected value. The old and new fiducials may be accepted by the system as accurate when they work simultaneously for some or all positions of the lower limb.

If the refined model includes the new fiducial, the navigation procedure may include determining if an estimated error in the calculated location of the bone segment is within an acceptable error range (block 820). This may be performed by the computer 18 estimating an expected error of the calculated current position of the bone segments based on the initial model and the refined models. The estimation may be performed according to various methods and/or may be based on various parameters. If the estimated error in the calculated position of the bone segment is considered to be unacceptable, for example by exceeding a predefined maximum error threshold for the bone segment, the navigation procedure may include providing a notification to the user, such as with a warning message, error message, and/or ending the navigation procedure (block 822). If, however, the estimated error is considered to be acceptable, such as by being within the predefined maximum error threshold, the new fiducial may be accepted together with its relationship to the old fiducials and tracked bone segment, based on the refined model estimations and calculations as explained above (block 824).

Once data from the refined model relative to the available fiducials 14 has been calculated by the computer 18 and stored in the database 36 as part of the implemented navigation procedure, the tracking system 40 may display automatically at certain steps of the procedure (or alternatively when requested by the user) information regarding the effect on the refined model that would be obtained after removing one or more of the accepted fiducials 14.

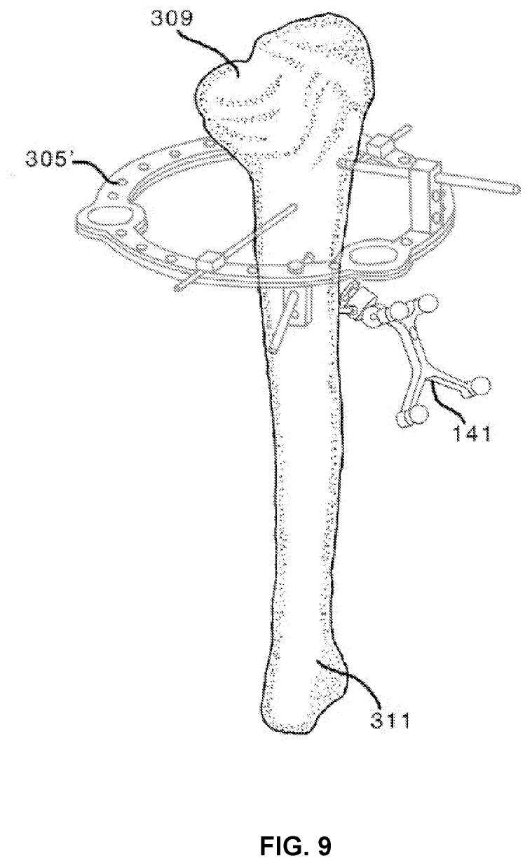

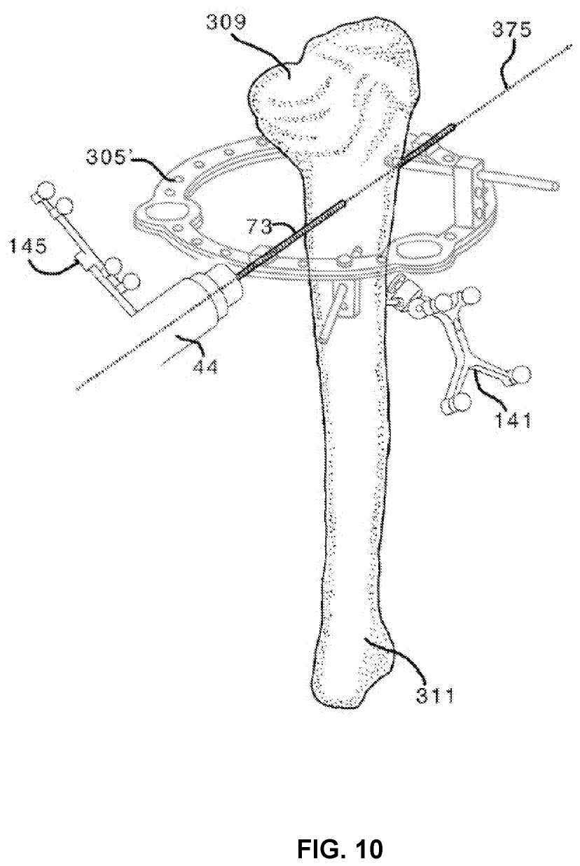

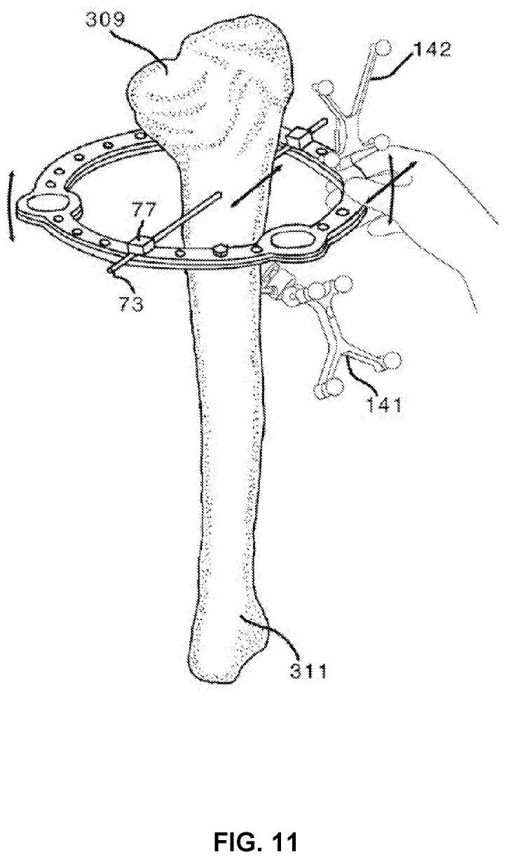

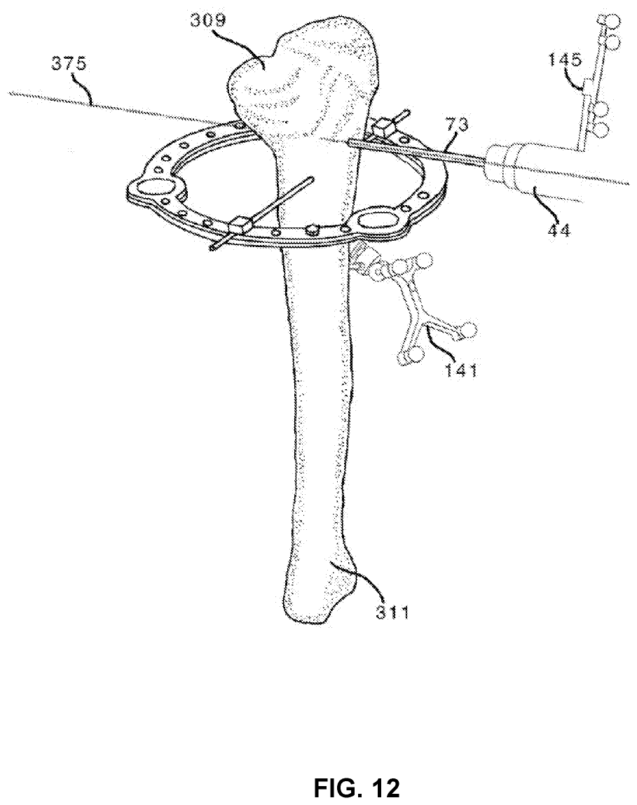

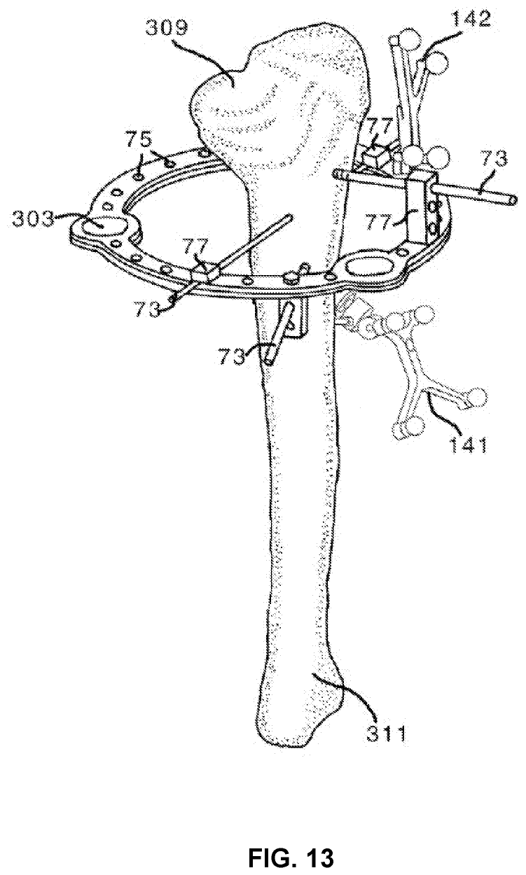

FIGS. 9 to 16 show a perspective view of the operative setting described in FIG. 1. After the initial fiducial 141 has been attached to the bone as shown in FIG. 9, the virtual position of the first base member 305′ of FIGS. 4-7 is shown over the tracked bone segment 309 (soft tissues not depicted), with a selected degree of opacity, as an example of an image rendered by the computer 18 on the display 24. Some of the features in FIG. 9 may be the same as or similar to some of the features in the other FIGs. described herein as noted by same and/or similar reference characters, unless expressly described otherwise. Additionally, reference may be made to features shown in any of the other FIGs. described herein and not shown in FIG. 9. In FIG. 10, a drill motor 44 with an attached fiducial 145 is used to insert transosseous fixations 73 through a pre-determined virtual trajectory 375 (e.g. as rendered in display 24), in accordance with an accepted virtual plan, in this example starting with a wire in an approximately lateral-medial direction, with a virtual first base member 305′ superimposed in its pre-selected final place. Some of the features in FIG. 10 may be the same as or similar to some of the features in the other FIGs. described herein as noted by same and/or similar reference characters, unless expressly described otherwise. Additionally, reference may be made to features shown in any of the other FIGs. described herein and not shown in FIG. 10. As shown in FIG. 11, the real base member 305 (with a fiducial 142 attached in a predefined position for proper tracking) may be secured to the wire with fixator clamps 77 or other similar mechanisms. Some of the features in FIG. 11 may be the same as or similar to some of the features in the other FIGs. described herein as noted by same and/or similar reference characters, unless expressly described otherwise. Additionally, reference may be made to features shown in any of the other FIGs. described herein and not shown in FIG. 11. With only one wire attachment to bone, the surgeon may be able to move the base member 305 in certain planes to place it more accurately in the preplanned position 305′, according to the real-time information provided by the navigation system 30. As shown in FIG. 12, a motor drill 44 attached to a fiducial 145 may be used to insert a half-pin 73 using the pre-determined virtual trajectory 375 rendered to the display 24. Some of the features in FIG. 12 may be the same as or similar to some of the features in the other FIGs. described herein as noted by same and/or similar reference characters, unless expressly described otherwise. Additionally, reference may be made to features shown in any of the other FIGs. described herein and not shown in FIG. 12. As shown FIG. 13, another half-pin 73 may be used to secure the first base member 305 in the desired position according to the virtual plan 305′. Some of the features in FIG. 13 may be the same as or similar to some of the features in the other FIGs. described herein as noted by same and/or similar reference characters, unless expressly described otherwise. Additionally, reference may be made to features shown in any of the other FIGs. described herein and not shown in FIG. 13. The first base member 305 may be fixed, and a fiducial 142 may be attached to a predetermined position to the first base member 305, (e.g. to one of the spaced apertures 75 and/or via a sheath or cover engaged with the base member 305, the sheath or cover having a set of radio-opaque fiducials attached thereon). Different fiducials may be configured differently for each base member type in the computer 18 by the tracking system 40. Different fiducial types may be selected through the computer 18 through preoperative or intraoperative input to identify the specific base member type that is being tracked and the precise position of the fiducial relative to the base member. For example, the base member 305 and its relation to the attached fiducial 142 may be identified by knowing the specific spaced apertures 75 where the fiducial 142 is located relative to an identifiable structure, such as split-ball connectors, tabs, or pre-planned specific strut position, for example according to the right-hand rule used to determine rotational motion.

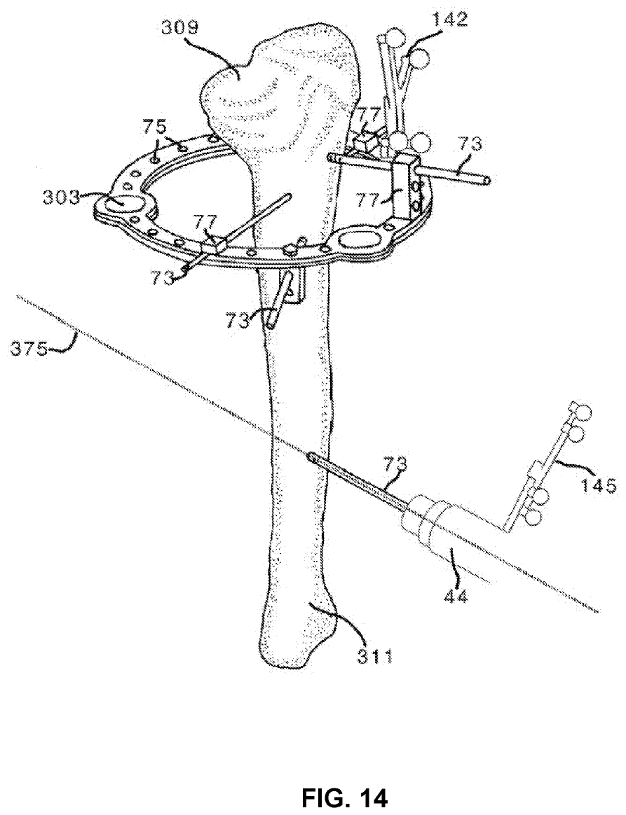

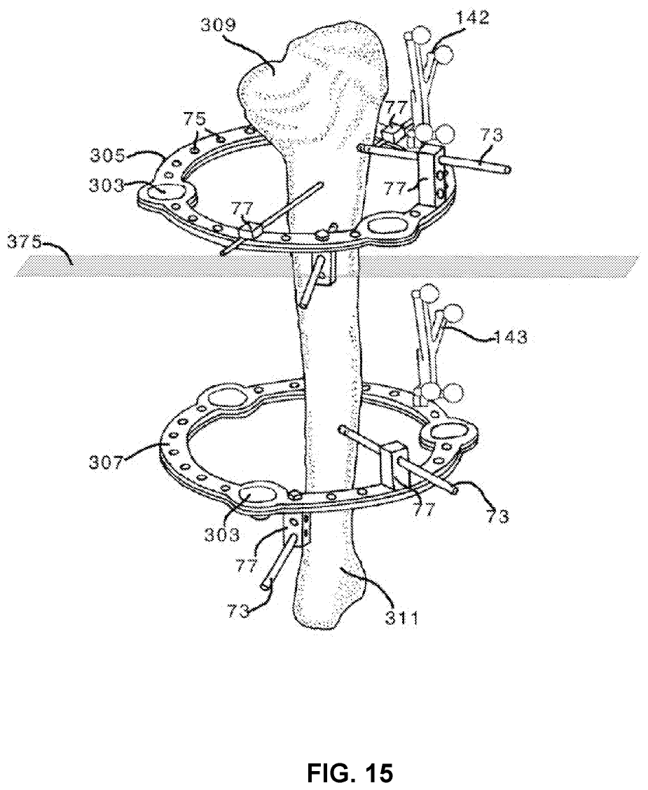

The computer 18 may perform the steps of the navigation procedure defined by block 800 automatically and/or upon receiving an instruction to perform on or more of the steps, such as from the surgeon. If the new fiducial 142 attached to the base member 305 is accepted by the navigation system (e.g. initially or if the refined model does not require both fiducials to work at the same time), the initial fiducial 141 may be removed, leaving only a fiducial 142 attached to the base member 305 for tracking of the target bone 10 (e.g. as shown in FIG. 14). Some of the features in FIG. 14 may be the same as or similar to some of the features in the other FIGs. described herein as noted by same and/or similar reference characters, unless expressly described otherwise. Additionally, reference may be made to features shown in any of the other FIGs. described herein and not shown in FIG. 14. A drill motor 44 with a fiducial 145 attached may be used to insert a half-pin 73 in a pre-determined virtual trajectory 375. The half-pin 73 may be used to secure the second base member to the second bone segment 311. As shown in FIG. 15, both base members 305, 307 may be secured to their respective bone segments 309, 311. Some of the features in FIG. 15 may be the same as or similar to some of the features in the other FIGs. described herein as noted by same and/or similar reference characters, unless expressly described otherwise. Additionally, reference may be made to features shown in any of the other FIGs. described herein and not shown in FIG. 15. Block 800 may be repeated for the fiducial 143 attached to the second base member 307. If accepted by the navigation system (e.g. initially or if the refined model does not require both fiducials 142, 143 to work at the same time), the target bone may be cut with a saw motor with an attached fiducial at the pre-selected site with help of a virtual trajectory 375, turning the target bone 10 effectively into two independent bone segments, 309 and 311. Subsequently, each bone segment may be tracked by its own fiducial 142, 143. The cut may be done once the struts are in place and secured, or it may be done before positioning the struts. If one fiducial attached to a base member may not work separately to track the bone segment correctly, further fixation elements, base members, and/or another fiducial may be attached to the specific bone segment, and the navigation procedure at block 800 may be repeated, until a proper configuration is found for both bone segments 309, 311 to be tracked separately. Alternatively, the cut may be performed first, re-setting or re-registering the navigation system 30 according to a multiple bone technique. A new fiducial may be attached to any part of the corrective device 11, such as any part of the base members 305, 307, or any Steinmann pin, half-pin, or any other kind of metal rod or connector attached to the bone or connecting other parts of the corrective device 11, such as screws or struts, in any position selected preoperatively and/or intraoperatively, automatically and/or with user input. After being accepted, the position of any of these tracked parts of the corrective device 11 may be registered in the navigation system 30 relative to the target bone 10 or fragment of it.

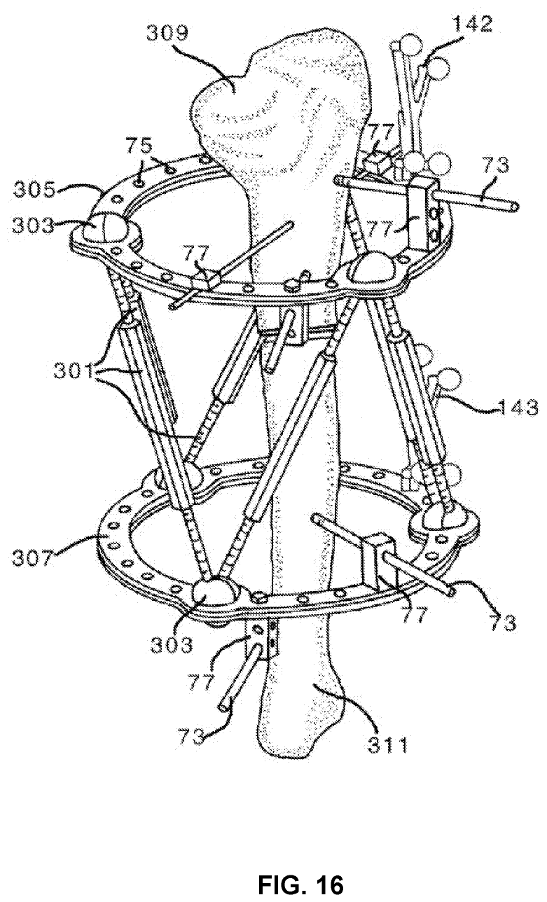

As shown in FIG. 16, the struts 301 may be fixed in place as planned. Some of the features in FIG. 16 may be the same as or similar to some of the features in the other FIGs. described herein as noted by same and/or similar reference characters, unless expressly described otherwise. Additionally, reference may be made to features shown in any of the other FIGs. described herein and not shown in FIG. 16. Knowing the distance for correction and a biologically safe velocity, the total number of days to safely correct the deformity may be determined. The rate may be, for example, 1 mm/day. The rate may be selected depending on factors relevant to a specific case. The movement of the independent bone segments 309, 311 may be followed with the fiducials 142, 143 attached to each base member 305, 307.

The fiducials 142, 143 may be removed in the operating room and later reattached to the predefined spaced aperture 75 or to any other part of the corrective device 11 registered in the navigation system 30, such as any other part of the base members 305, 307, Steinmann pin, half-pin, or any other kind of metal rod or connector, such as a screw or strut. The fiducials may be reattached in consultations and/or during control X-rays. The navigation system 30 may be used to track the movement of both bone segments 309, 311. The surgeon may repeat the navigation procedure at the end of the corrective procedure to the target bone using a multiple bone technique. Intraoperative fluoroscopic projections may be repeated with an imaging device 20 attached to a fiducial 14, to re-register the base members 305, 307 relative to the bone segments 309, 311, in order to obtain a refined model for the follow-up of the treatment. Blocks 800, 816, and/or 820, may be followed by the computer 18 to accept or reject the new model obtained with the same fiducials 14 on separated bone segments 309, 311, or to reject them and work with the initial models obtained with joint bone segments 309, 311, in accordance with the multiple bone technique. The surgeon may use the navigation system 30 for periodic follow-up consultations, instead of X-rays. The surgeon may control follow-up with a tracked imaging device, such as an imaging device 20 with a fiducial 14 attached, to restart the navigation procedure and/or to test the accuracy of the navigation system 30.

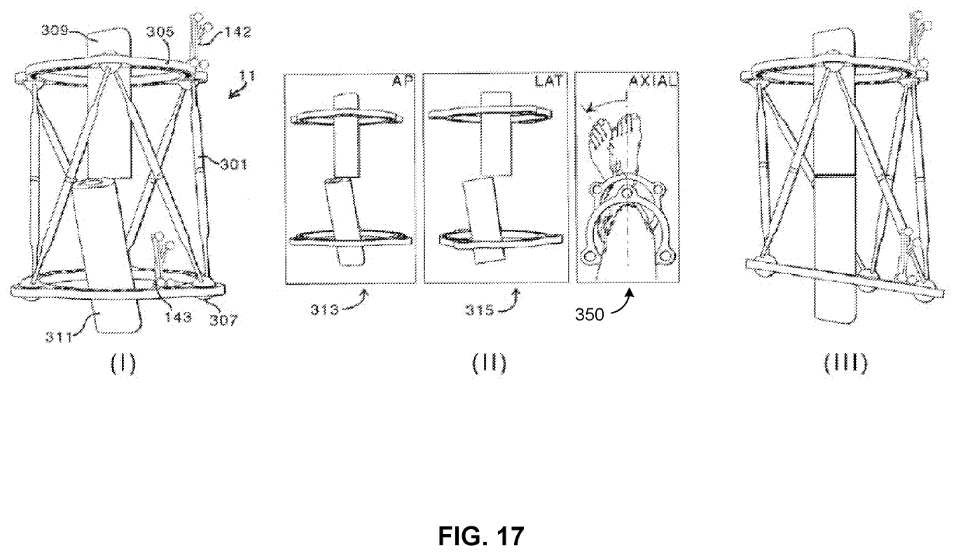

The multiple bone technique may include an acute setting (for example fractures or dislocations), or a use for chronic settings including two or more bone segments, such as joint-spanning external fixators (for example arthrodiastasis or foot deformity corrections). It may also be used for single bone corrections when two or more markers are used, at least one on each planned bone segment, before or after performing the planned osteotomy or osteotomies. In accordance with the multiple bone technique, the acute technique for a tibial fracture is illustrated in FIG. 17 as an example. Alternative uses of the multiple bone technique may also be used. Some of the features in FIG. 17 may be the same as or similar to some of the features in the other FIGs. described herein as noted by same and/or similar reference characters, unless expressly described otherwise. Additionally, reference may be made to features shown in any of the other FIGs. described herein and not shown in FIG. 17.

The surgeon may attach at least one fiducial 142, 143 to each bone segment 309, 311, and obtain intraoperative images with an imaging device 20 attached to a fiducial 14, to create a model 204 in accordance with the navigation procedure. For example, the intraoperative images (such as AP and LAT fluoroscopic images, intraoperative CT scan, etc.) may be registered with the real movements of the target bones. The fiducials 142, 143 may be attached on outer surfaces of the bone segments 309, 311, or may be implanted into the bone segments 309, 311. The imaging device 20 may not have a fiducial 14 attached. Navigation may be done with a selection of points in the X-rays corresponding to the fiducial 14 and pre-selected anatomical landmarks, in a process which may be partially or fully automated by the computer 18 with appropriate image classification algorithms, with or without user input, and which may include the use of a tracked probe 26. Registered intraoperative images may be in turn registered to other preoperative or intraoperative images. For example, intraoperative fluoroscopy may be registered with preoperative CT scan of the fracture. An intraoperative image (fluoroscopy or CT scan) may be registered with preoperative planned image (fluoroscopy or CT scan) or with: intraoperative or preoperative images of the contralateral healthy bone (in this case the contralateral tibia) as a target for correction; planned anatomical and/or mechanical axes 206 in the AP and/or LAT views; and/or a rotation determined from a clinical examination. Once both bone segments of the target bone 10 are tracked by the tracking system 40, the surgeon may select the appropriate size corrective device at using virtual trials, with or without using real base members tracked by fiducials 14 over the real target bone 10. The surgeon may complete the first stage of surgery without navigation, selecting the appropriate device size and attaching both base members 307, 309 to their respective bone segments 309, 311. Fiducials 142, 143 may be attached to each base member 307, 309 in the predefined position, such as a specific space aperture 75. Tracking of bone segments 309, 311 may be done by obtaining intraoperative images, for example AP and LAT fluoroscopic images as explained above, and then tracking the bone segments 309, 311 through their attached base members 307, 309.

Once both bone segments 309, 311 are tracked (e.g. as shown in element (I) of FIG. 17), the surgeon may use the navigation system 30 to reduce the fracture before or during application of the external fixator 11 to the extent that it is intended or possible. In certain types of fractures, as well as in chronic pathologies including multiple bones (such as complex foot deformity corrections), it is not possible to move bones to the intended final position. Due to the 6-axis correctability of hybrid external fixators, residual deformities (e.g. as shown in the views of element (II) in FIG. 17) may be corrected later by adjusting struts gradually, eliminating the need for subsequent anesthesia or corrective device modification. As in the single bone technique, the process to obtain the final correction (e.g. as shown in element (III) of FIG. 17) may include 1) obtaining deformity parameters of the first bone segment 309 relative to the second bone segment 311; 2) selecting an origin at the deformity site that will act as a reference point, such as at the same level that the AP and LAT displacements are measured; 3) measuring device eccentricities once a base member is selected as the first or moving one, including a) vertical distance from the first base member to the origin, b) horizontal displacement of the origin from the centerline of the device (AP displacement and LAT displacement), and c) predetermined orientation of the device and the amount the second bone segment is rotated about its axis from its correct position; and/or 4) calculating the effective length of each strut 301 to configure the corrective device 11 to mirror the deformity.

Relevant clinical cues may be rendered by the navigation system 30 to the display 24 during the preoperative or intraoperative planning, depending on the specific anatomic area and pathology treated. For example, the cues may include visual or sound alarms when a base member will lie too close to the soft tissues, which may include taking into account flexion-extension and rotation of adjacent joints. The computer 18 may suggest different kinds of appropriate base members for each anatomical area or soft tissue diameter, such as half-rings or arches for the upper thighs and arms. During the attachment of transosseous fixations, the computer 18 may render to the display 24 anatomical structures at risk in the intended path, depending on the selected level, such as neurological or vascular structures displayed as virtual images overlaid over the real or virtual images used in real time for navigation. Similarly, the navigation system 30 may take into account the risk of transfixation pin-induced joint stiffness, for example by signaling to the user (e.g. the surgeon, nurse, and so forth) the best areas with minimum soft-tissue displacement for possible movement of adjacent joints, or the need to change the position of the joint during insertion to create a “store” of soft tissue by inserting it through the “flexor” and “extensor” surfaces of the segment. The computer 18 may notify the surgeon to perform the final bone cuts, including cuts of adjacent bones that may limit the planned correction, such as the fibula for the tibia, or the ulna for the radius. The computer 18 may offer cues as to the optimal adjustment amounts of the corrective device, depending on the position of the moving bone relative to circumambient soft tissue, vessels and nerves, with or without further input by the user depending on reported pain by the patient and/or other considerations.

The computer 18 may render to the display 24 technical cues to help achieve the best possible configuration and fixation, including images explaining how to use appropriate techniques suited for the specific case. For example, the computer 18 may suggest during surgery as the preplanned steps progress, in order to increase the stability of the corrective device 11: to increase the number of transosseous fixations; to increase the distance between the level of insertion of first and second base members; to increase the level of insertion of the base member and stabilizing transosseous elements; to increase or decrease the angle for wires crossing in the support; and/or to increase the distance and angles to insert the wires or half-pins from the support. The computer 18 may suggest the optimum number of basic and support members, as well as the optimum number of connecting elements between them, such as to avoid angular deformation due to an eccentric effect (eccentric distension or compression). The computer 18 may suggest the use of: bent wires and/or wires with stops for repositioning in fractures; and/or half-pins as “pusher” or “puller” elements. The computer 18 may notify the user when basic transosseous elements are not perpendicular to the bone's long axis such that, when changing the spatial orientation of the bone fragment with a repositioning/fixation wire (or half-pin), a Z-like deformation of the basic wires may occur, with the corresponding force induced by elastic deformation. The computer 18 may notify the surgeon that moving the transosseous fixation from its position on the base member, for example changing spaced apertures 75 and/or specific fixator clamp 77 type, may help reduce fragments in a particular direction and distance. The computer 18 may include different design concepts that suit the specific case under treatment, with an evaluation of alternatives, for example based on angles of strut-ring, displacement, safety factor, and tolerance joint fitting, with estimations of compression, bending, and torsional stability which can be based on integrated finite element analysis program, whereby the computer 18 may rank each alternative configuration based on the user's preferences or on predefined values, adapting them in real time depending on the user's selection during surgery. The computer 18 may also present different distributions of adjustment amounts, instead of only linear adjustments of struts, depending on the nonlinear relation between strut lengths and platform pose. The computer 18 may present different deformity correction algorithms based on the motion trajectory of the specific external fixator 11 used, including clinical considerations and personal preferences.

The navigation system is described above regarding a specific type of hybrid external fixator with specific components and fiducials 14 applied to the target bone 10. The navigation system and method may be applied to any type of external fixation system using any types of components, any of which may be tracked using the navigation system. Such external fixators may include those used for acute or chronic pathology of bones or soft tissues of any part of the body, including bodies of non-human animals. Similarly, the navigation method and system described may be used for other types of surgery such as internal fixation devices or percutaneous devices.

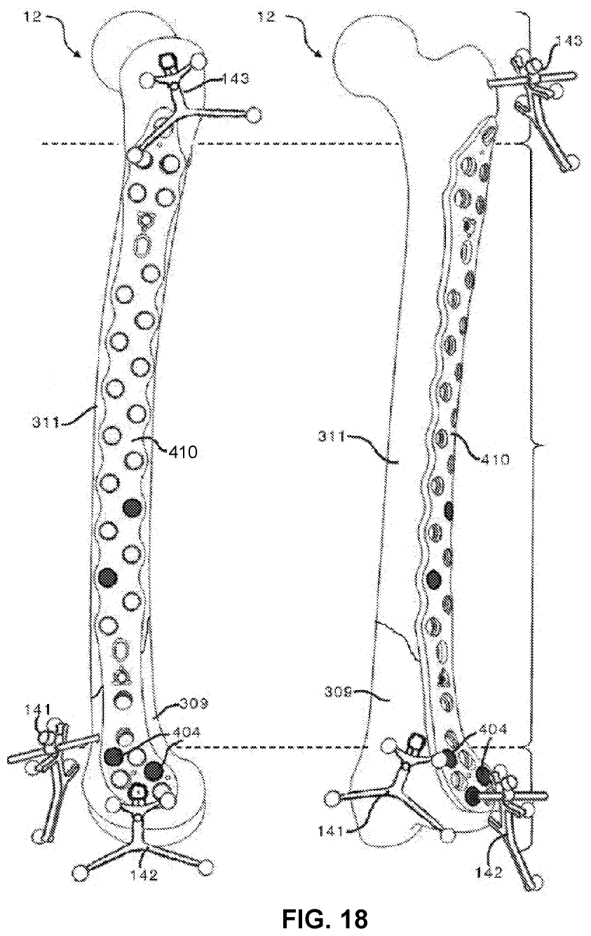

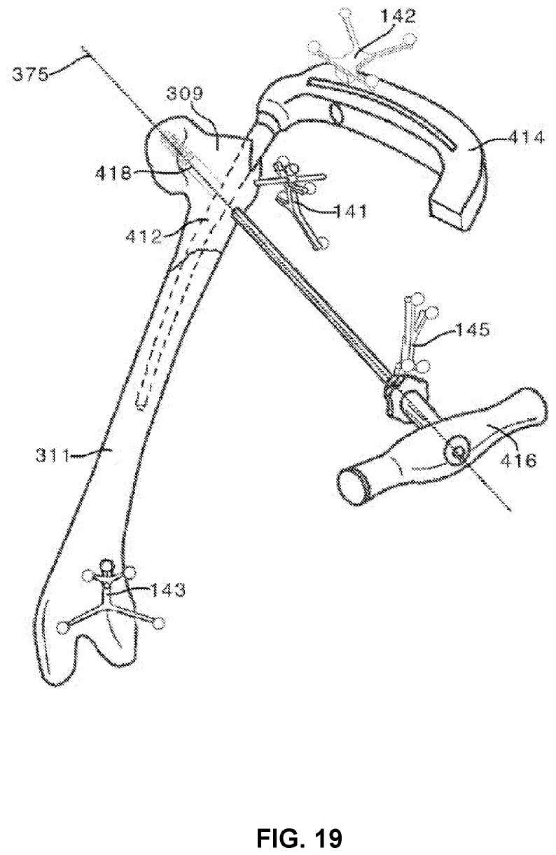

FIGS. 18 and 19 illustrate a percutaneous surgery according to the multiple bone technique of the navigation system, as performed on a fracture of the femur 12. A fiducial 141, 143 is attached first to the two bone fragments 309, 311, and at least two perpendicular X-ray views are taken to register each fiducial 141, 143 to the respective views of each fragment 309, 311, including AP and LAT views (an oblique position may provide a lateral-medial fluoroscopic view of the proximal aspect of the femur). The registration may include reconstructing a 3D model, as explained above for the multiple bone technique. Registration and/or creation of a 3D model may include imaging of the contralateral (healthy) bone. Once the bone 12 is registered, the fracture may be accurately reduced without repeated intraoperative fluoroscopic images.

In FIG. 18, a fracture of the distal aspect of the femur 12 is shown, treated with a long plate 410 attached to a fiducial 142 with a screw. Some of the features in FIG. 18 may be the same as or similar to some of the features in the other FIGs. described herein as noted by same and/or similar reference characters, unless expressly described otherwise. Additionally, reference may be made to features shown in any of the other FIGs. described herein and not shown in FIG. 18. The plate may also be inserted with a percutaneous handle, and the fiducial 142 may then be attached to the handle. After insertion of distal screws 404 for a rigid fixation of the plate to the distal bone fragment 309, stability and reliability of the new fiducial 142 may be tested according to the navigation procedure, and the new fiducial may be accepted (e.g. as described regarding block 812 or 824), in which case the fiducial 141 of the distal fragment may be removed when necessary to continue surgery. The navigation system 30 may render virtual trajectories for the percutaneous insertion of additional screws 404 without other mechanical systems, and the 3D model of the bone fragments 309, 311 may be rendered in real time to the display 24 to help correct the final relative position of the bone segments 309, 311 during plate fixation.

In FIG. 19, a fracture of the proximal aspect of the femur 12 is shown. Some of the features in FIG. 19 may be the same as or similar to some of the features in the other FIGs. described herein as noted by same and/or similar reference characters, unless expressly described otherwise. Additionally, reference may be made to features shown in any of the other FIGs. described herein and not shown in FIG. 19. After the fracture is reduced, the intramedullary nail 412 may be inserted using a handle 414 attached to a fiducial 142. When enough stability is obtained between the inserted nail 412 and the proximal bone fragment 309 or fragments 309, 311 according to the navigation procedure 200, and the new fiducial 142 has been accepted, the fiducial 141 of the proximal fragment may be removed, continuing navigation, for example with the insertion of cephalic screw or screws 418 and locking screws with help of appropriate handles 416 attached to fiducials 142 which may render the pre-determined virtual trajectory 375 to the display 24, without a need for traditional mechanical guides.

In fractures with more than two fragments, the navigation system may be used with one fiducial 14 attached to each fragment. The navigation system may be used to reduce and/or fix fragments in pairs until two separated bone segments remain. The final pair may also be fixed using the navigation system.

Once a fracture is reduced and/or provisionally fixed and a stable bone-fiducial relationship is obtained, both fiducials 141, 143 attached to the bone fragments 309, 311 may be removed, and navigation may be continued with the fiducial or fiducials 142 attached to the device. A surgery may be performed with reduction done without navigation, and may be completed with an imageless navigation system, wherein a fiducial 142 is attached the percutaneous device, for example a percutaneous nail handle or a percutaneous plate handle. With a known fiducial-device relationship, the navigation system 30 may offer the appropriate trajectories for screws without a need for mechanical guides, allowing for a handle of a reduced size.

While the foregoing written description may enable one of ordinary skill to make and use the navigation systems and methods specifically described, those of ordinary skill will understand and appreciate the existence of variations, combinations, and equivalents of the specific systems, methods, and examples herein. Thus, the disclosure should not be viewed as limited to the specifically described systems, methods, and examples.

What is claimed:

1. A system comprising:
a display device;
a first fiducial configured to be attached to a target bone of a patient, wherein the target bone comprises a first fragment and a second fragment; and
a computing system coupled to the display device, the computing system being configured to:
receive preoperative image information or intraoperative image information of the target bone;
register the first fiducial in a common coordinate system;
register the preoperative image information or the intraoperative image information of the target bone in the common coordinate system;
register the target bone in the common coordinate system as the first fiducial is attached to the first fragment of the target bone;
generate a two-dimensional (2D) virtual representation or three-dimensional (3D) virtual representation of:
the preoperative image information or the intraoperative image information;
an operative instrument; or
a corrective device;
register the 2D virtual representation or 3D virtual representation in the common coordinate system;
generate a 2D virtual representation or 3D view configured to be displayed by the display based on the 2D or 3D virtual representation;
during an osteotomy, track movement in the common coordinate system from a first position to a second position of:
the target bone;
the operative instrument; or,
the corrective device;
register, in the common coordinate system, a second fiducial as the second fiducial is attached to the second fragment of the target bone;
create a refined model of the target bone based on the second fiducial; and
calculate or adjust a deformity parameter of the target bone based on the refined model of the target bone.

2. The system of claim 1, wherein:
the preoperative or intraoperative image information is 2D; and
the computing system is further configured to generate a 3D image from the preoperative or intraoperative image information using a 2D reconstruction to a 3D reconstruction.

3. The system of claim 1, wherein the computing system is further configured to track the movement of the target bone by tracking a position of the first fiducial or the second fiducial in real time.

4. The system of claim 1, wherein the computing system is further configured to compare, in real time, tracking data of the operative instrument or the corrective device to a tracked position of the target bone during surgery.

5. The system of claim 1, wherein the computing system is configured to track, in the common coordinate system, a position or an orientation of:
the target bone;
an imaging device;
the operative instrument;
the corrective device;
a person participating in the osteotomy; or
the display device.

6. The system of claim 5, wherein the computing system is configured to track using:
an attached passive optical marker;
an attached active optical marker;
magnetic tracking;
electromagnetic tracking;
ultrasonic tracking;
mechanical tracking;
inertial measurement; or
a 3D scanner.

7. The system of claim 1, wherein the preoperative or intraoperative image information of the target bone is registered with a structure of the target bone using a navigation system.

8. The system of claim 1, wherein:
the display device comprises:
an electronic display screen; or
an optical see-through head mounted device attached to a third fiducial registered in the common coordinate system;
the computing system is further configured to generate and display, by the display device:
a 2D view of the 2D or 3D virtual representation of:
the operative instrument; or
the corrective device; or
a 3D stereoscopic view of the 2D or 3D virtual representation of:
the operative instrument; or
the corrective device.

9. The system of claim 8, wherein the computing system is further configured to:
merge, for a common display, the 2D view or the 3D view with the preoperative or intraoperative image information of the target bone; or
superimpose, in an augmented reality environment, the 2D view or the 3D view onto the preoperative or intraoperative image information of the target bone.

10. The system of claim 1, wherein the preoperative or intraoperative image information comprises:
a 2D image of the target bone;
a computed tomography image of the target bone;
a magnetic resonance image of the target bone; or
an ultrasound image of the target bone.

11. The system of claim 1, wherein the corrective device comprises:
a hybrid external fixator;
a circular external fixator;
a monolateral external fixator;
an intramedullary device;
an internal fixation device; or
an external fixation device.

12. The system of claim 1, wherein the at least one computing system is further configured to merge with or superimpose on the preoperative or intraoperative image information of the target bone one or more graphical representations of:
the operative instrument;
the corrective device;
a surgical guide;
a surgical technique; or
an anatomical model.

13. The system of claim 1, wherein the computing system is further configured to:
detect changes in rotation, translation, or lateralization of:
the target bone;
the operative instrument; or
the corrective device; and
adjust the 2D or 3D virtual representation, based on the detected changes, of:
the preoperative or intraoperative image information of the target bone;
the operative instrument; or
the corrective device.

14. The system of claim 1, wherein the computing system is further configured to calculate the deformity parameter of the target bone based on:
an anatomical landmark on the preoperative or intraoperative image information that is automatically-selected or user-selected; and
a corresponding anatomical landmark on the target bone that is automatically-selected or user-selected.

15. The system of claim 14, wherein the deformity parameter comprises an automatically-selected or user-selected origin on the target bone of the osteotomy.

16. The system of claim 1, wherein computing system is further configured to calculate:
an effective diameter of a first base member;
an effective diameter of a second base member;
an initial neutral length of a strut extending between and connected to the first base member and the second base member;
a position of a deformity of the target bone with respect to the first base member or the second base member; and
an adjustment of the first base member, the second base member, or the strut to at least partially correct the deformity.

17. The system of claim 1, wherein:
the computing system is further configured generate, during the osteotomy, a display of an operative parameter to be shown by the display device, the operative parameter comprising:
the deformity parameter;
an adjustment of the deformity parameter;
a corrective device parameter; or
a mounting parameter of the corrective device; and
the display comprises:
a graphical representation of the operative parameter; or
a numerical value of the operative parameter.

18. A method, comprising:
registering a first fiducial in a common coordinate system;
receiving preoperative image information of a first target bone or a second target bone;
registering the preoperative image information in the common coordinate system;
attaching the first fiducial to the first target bone;
registering the first target bone in the common coordinate system as the first fiducial is attached to the first target bone;
initiating an osteotomy on the first target bone or on the second target bone;
receiving intraoperative image information of the first target bone or the second target bone;
registering the intraoperative image information in the common coordinate system;
generating, during the osteotomy, virtual representations of:
the first target bone using the preoperative image information or the intraoperative image information;
the second target bone using the preoperative image information or the intraoperative image information;
an operative instrument; or
a corrective device;
registering any of the virtual representations in the common coordinate system;
displaying any of the virtual representations to an individual participating in the osteotomy;
attaching a second fiducial to the first target bone or the second target bone;
registering, in the common coordinate system, the second fiducial as the second fiducial is attached to the first target bone or the second target bone;
creating a refined model of the first target bone, the second target bone, or a joint between the first target bone and the second target bone; and
calculating or adjusting a deformity parameter of the first target bone, the second target bone, or the joint based on the refined model.

19. The method of claim 18, further comprising:
registering the corrective device in the common coordinate system;
calculating or adjusting, during the osteotomy, a position of the corrective device in the common coordinate system; and
calculating or adjusting a corrective device parameter or a mounting parameter of the corrective device on the first target bone or the second target bone.

20. The method of claim 18, further comprising:
tracking a pose change of:
the first target bone;
the second target bone;
the joint;
the operative instrument;
the corrective device;
the individual participating in the osteotomy; or
an optical see-through head mounted display;
generating an augmented reality display of any of the virtual representations;
displaying, by the optical see-through head mounted display, the augmented reality display, wherein:
the augmented reality display is superimposed on the first target bone, the second target bone, or the joint; and
the augmented reality display is adjusted based on the tracked pose change.
